(19) United States
(12) Patent Application Publication
(10) Pub. No.: US 2015/0094793 A1
(43) Pub. Date: Apr. 2, 2015

(54) FLUIDIC CONDUCTORS FOR IMPLANTABLE ELECTRONICS

(71) Applicant: Martin Joseph Svehla, Macquarie University (AU)

(72) Inventor: Martin Joseph Svehla, Macquarie University (AU)

(21) Appl. No.: 14/039,915

(22) Filed: Sep. 27, 2013

(51) Int. Cl. A61N 1/05

(52) U.S. Cl. CPC A61N 1/0541
USPC 607/137

(57) ABSTRACT

Fluidic conductors deliver electrical signals to targeted locations within an organ. Both the insulating materials and conductive media components of the fluidic conductors are ultra-flexible. The small size of the fluidic conductors makes the technology particularly applicable to auditory prostheses, for example, cochlear implants, that deliver electrical signals to very discrete locations within the cochlea.

### BACKGROUND

Implantable medical devices can deliver electrical charges or signals to specific targeted areas, typically neural structures, within a body tissue or organ. Electrical conductors include an outer insulating material that surrounds a conductive material, typically a wire or other solid conductive medium. Differences in thermal expansion characteristics, flexibility, surface roughness, robustness, and other material characteristics can often lead to failure of such conductors due to breakdown of either or both of the insulating material and conductive element.

### SUMMARY

Embodiments disclosed herein relate to fluidic conductors for electronics. The technologies disclosed herein have particular application in medical devices implanted within a bodily tissue (human, mammalian, or otherwise). Such devices include stimulating electrode arrays. However, any type of electronics requiring ultra-flexible electrical conductors also can benefit from these technologies. Such electronics can include those subject to excessive vibration or movement (due to, for example, articulation of machine parts or levers). The small size of the fluidic conductors makes the technology applicable to auditory prostheses, for example, cochlear implants, that deliver electrical signals to very discrete locations within the cochlea.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

The same number represents the same element or same type of element in all drawings.

FIG. 1 is a partial view of a behind-the-ear auditory prosthesis worn on a recipient.

FIG. 2 is a side view of an embodiment of an implantable portion of an auditory prosthesis.

FIG. 3 is a side view of another embodiment of an implantable portion of an auditory prosthesis.

FIG. 4A is a partial perspective view of an adapter body utilized in an internal component of an auditory prosthesis.

FIG. 4B is a partial perspective view of an intracochlear body utilized in an internal component of an auditory prosthesis.

FIG. 5 is a partial top view of another embodiment of an internal component of an auditory prosthesis.

FIG. 6A is a cross-sectional view of the adapter body of FIG. 5.

FIG. 6B is a cross-sectional view of the intracochlear body of FIG. 5.

### DETAILED DESCRIPTION

The technologies disclosed herein can be used in conjunction with various types of implantable electronics, or other electronics that require small and/or extremely flexible conductive pathways for the transmission of electrical signals. For clarity, however, the technology will be described in the context of an auditory prosthesis such as a cochlear implant that utilizes both an external portion and an implantable portion. Of course, one of skill in the art will appreciate that the flexible conductive pathways can also be utilized with totally implantable cochlear implants as well.

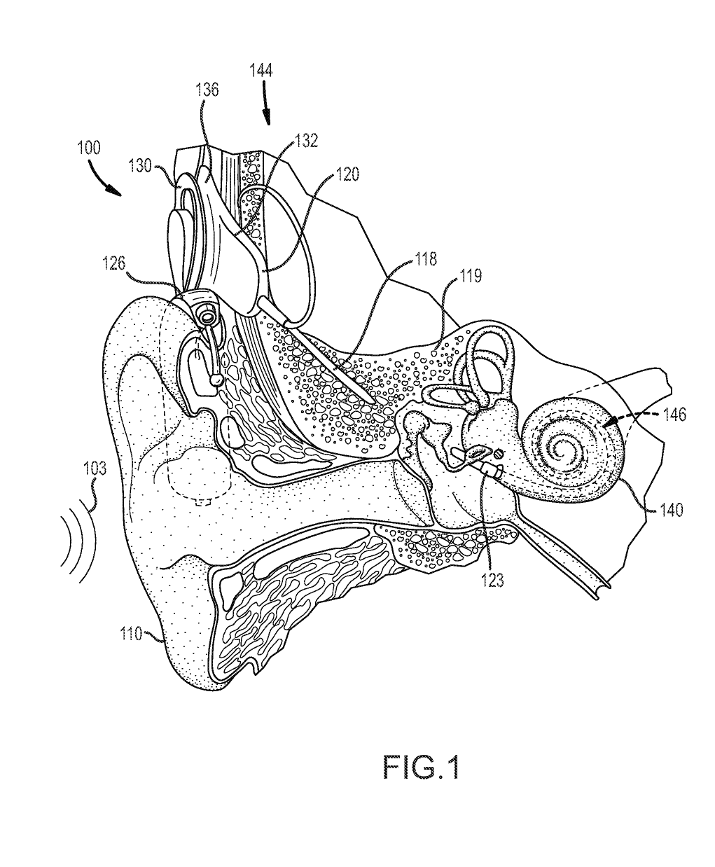

Referring to FIG. 1, cochlear implant system 100 includes an implantable component 144 typically having an internal receiver/transceiver unit 132, a stimulator unit 120, and an elongate lead 118. The internal receiver/transceiver unit 132 permits the cochlear implant system 100 to receive and/or transmit signals to an external device 126 and includes an internal coil 136, and preferably, a magnet (not shown) fixed relative to the internal coil 136. These signals generally correspond to external sound 103. Internal receiver unit 132 and stimulator unit 120 are hermetically sealed within a biocompatible housing, sometimes collectively referred to as a stimulator/receiver unit. The magnets facilitate the operational alignment of the external and internal coils, enabling internal coil 136 to receive power and stimulation data from external coil 130. Elongate lead 118 has a proximal end connected to stimulator unit 120, and a distal end implanted in cochlea 140. Elongate lead 118 extends from stimulator unit 120 to cochlea 140 through mastoid bone 119.

In certain examples, external coil 130 transmits electrical signals (e.g., power and stimulation data) to internal coil 136 via a radio frequency (RF) link, as noted above. Internal coil 136 is typically a wire antenna coil comprised of multiple turns of electrically insulated single-strand or multi-strand platinum or gold wire. The electrical insulation of internal coil 136 is provided by a flexible silicone molding. Various types of energy transfer, such as infrared (IR), electromagnetic, capacitive and inductive transfer, can be used to transfer the power and/or data from external device to cochlear implant. In the depicted embodiment, the implantable component 144 also includes an adapter 123 disposed outside of the cochlea 140. The adapter 123 and flexible conductors extending therefrom (that form a stimulating assembly 146) are described in further detail below.

There are a variety of types of intra-cochlear stimulating assemblies including short, straight and peri-modiolar. Stimulating assembly 146 is configured to adopt a curved configuration during and or after implantation into the recipient's cochlea 140. To achieve this, in certain arrangements, stimulating assembly 146 is pre-curved to the same general curvature of a cochlea 140. Such examples of stimulating assembly 146, are typically held straight by, for example, a stiffening stylet (not shown) or sheath which is removed during implantation, or alternatively varying material combinations or the use of shape memory materials, so that the stimulating assembly can adopt its curved configuration when in the cochlea 140. Other methods of implantation, as well as other stimulating assemblies which adopt a curved configuration, can be used.

Stimulating assembly can be a perimodiolar, a straight, or a mid-scala assembly. Alternatively, the stimulating assembly can be a short electrode implanted into at least in basal region. The stimulating assembly can extend towards apical end of cochlea, referred to as cochlea apex. In certain circumstances, the stimulating assembly can be inserted into cochlea via a cochleostomy. In other circumstances, a cochleostomy can be formed through round window, oval window, the promontory, or through an apical turn of cochlea.

As apparent from the above description, it is important that the internal components of the cochlear implant display flexibility. This is especially desirable for the components that are subject to bending stress (e.g., the stimulating assembly 146) or that must flex due to recipient movement (e.g., the elongate lead 118). Often, the flexibility of a particular component can be limited by the materials that are utilized in the manufacture of said component. In known cochlear implants, fiber optics or conductive wires that conduct signals from the stimulator unit 120 are often significantly less flexible than the plastic or silicone bodies or sheathing in which those components are contained. Accordingly, the technologies described further below utilize conductive fluids or other highly deformable conductive media to deliver electrical signals from the stimulator unit 120 to a contact array disposed within the cochlea 140.

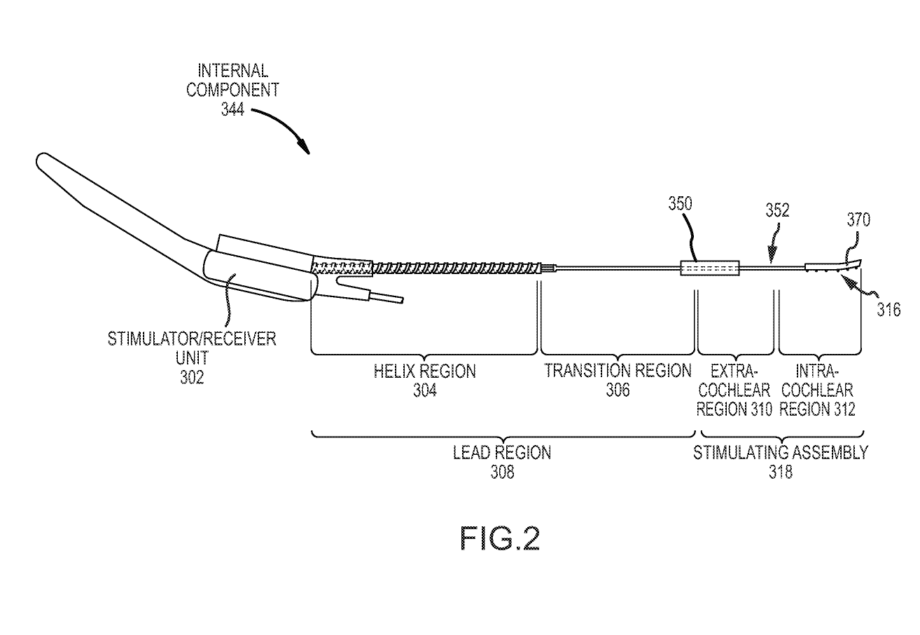

FIG. 2 is a simplified side view of an internal component 344 having a combined stimulator/receiver unit 302 that receives encoded signals from an external component of the cochlear implant system. Internal component 344 terminates in a stimulating assembly 318 that includes an extracochlear region 310 and an intracochlear region 312. Intracochlear region 312 is configured to be implanted in the recipient's cochlea and has disposed thereon a contact array 316. Each discrete contact in the array 316 is operatively connected to the stimulator/receiver unit 302 as described below.

Internal component 344 further includes a lead region 308 coupling stimulator/receiver unit 302 to stimulating assembly 318. Lead region 308 includes a region 304 which is commonly referred to as a helix region, however, the required property is that the lead accommodate movement and is flexible, it does not need to be formed from wire wound helically. Lead region also comprises a transition region 306 which connects helix region 304 to stimulating assembly 318. Electrical stimulation signals generated by stimulator/receiver unit 302 are delivered to contact array 316 via lead region 308. Helix region 304 prevents lead region 308 and its connection to stimulator/receiver 302 and stimulating assembly 318 from being damaged due to movement of internal component 344 (or part of 344) which can occur, for example, during mastication.

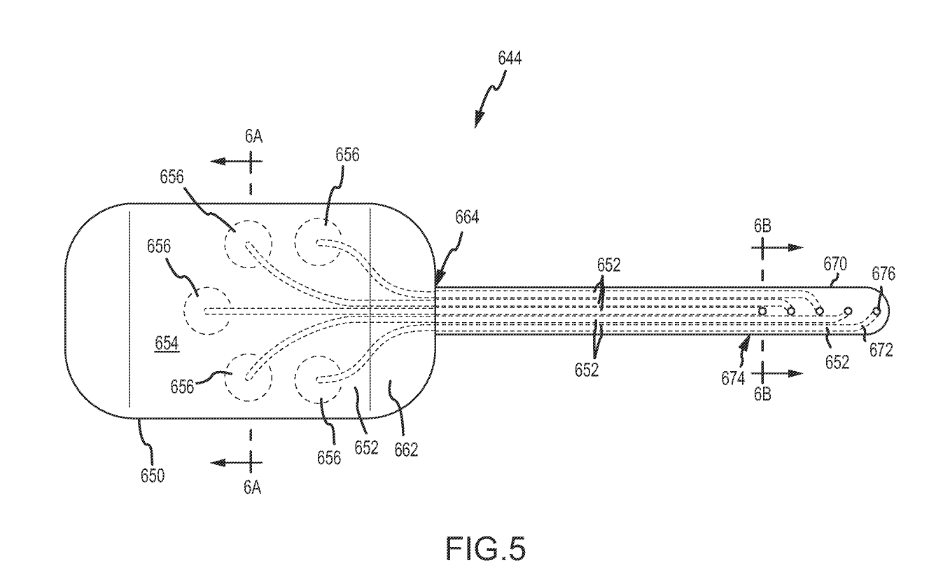

The extracochlear region 310, in this embodiment, includes an adapter body 350 that contains a plurality of electrode contacts or other conductive element termination points (described below). The adapter 350 is connected to the stimulator/receiver unit 302 via the lead region 308 and the structures and components included therein. Each electrode contact is connected to a wire or other conductive element so as to be operatively linked to the stimulator/receiver unit 302. A plurality of microtubes 352 extend from the adapter 350 to the intracochlear region 312. Individual microtubes 352 can be bound together or discrete from adjacent microtubes 352. In embodiments where the microtubes are discrete from each other, each microtube may move as required, with minimal, if any, effect on the movement of adjacent microtubes. The ends of the microtubes 352 form a contact array 316 in the intracochlear region 312 that delivers electrical signals to locations within the cochlea. The contact array 316 is disposed in an intracochlear body 370 that is inserted into the cochlea. In an alternative embodiment, the adapter body 350 and intracochlear body 370 can be an integral component (as depicted, for example, in FIG. 5).

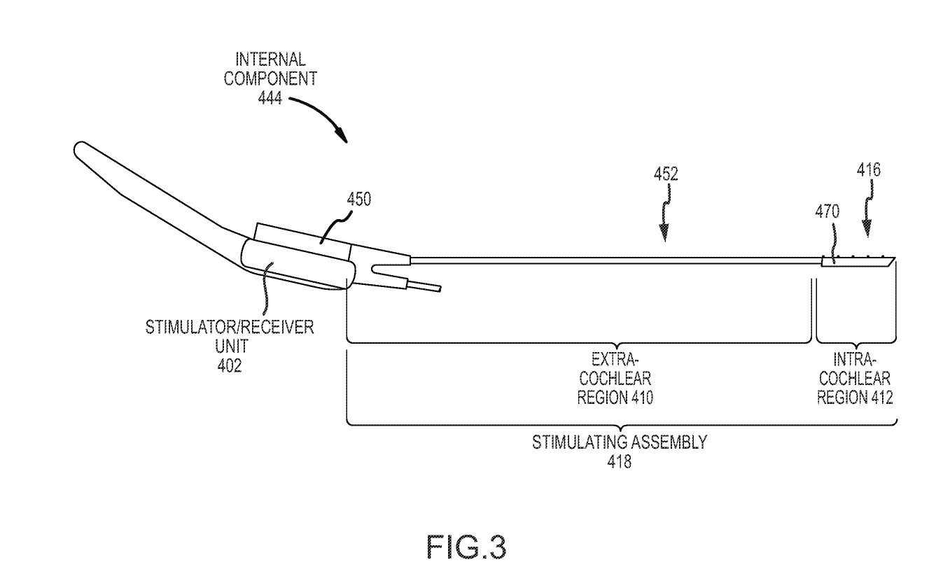

FIG. 3 depicts another embodiment of an internal component 444 for use with a cochlear implant system. Certain of the components utilized in the embodiment of FIG. 2 are not described again, unless otherwise noted. In this embodiment, an adapter body 450 containing a plurality of electrode contacts or conductive element termination points is fixed to a stimulator/receiver unit 402. The stimulating assembly 418 includes a plurality of elongate structures 452, which may be microtubes, extending from the adapter 450. The lengths of the structures 452 are determined as required or desired for a particular application. The elongate structures 452 can be bound together or discrete from adjacent elongate structures 452, as described above. The ends of the elongate structures 452 form a contact array 416 in the intracochlear region 412 that delivers electrical signals to discrete locations within the cochlea. The contact array 416 can be disposed within an intracochlear body 470 that is inserted into the cochlea. With these different internal assemblies in mind, the structure of the adapter bodies, elongate structures, and intracochlear bodies depicted in FIGS. 2 and 3 are described further below.

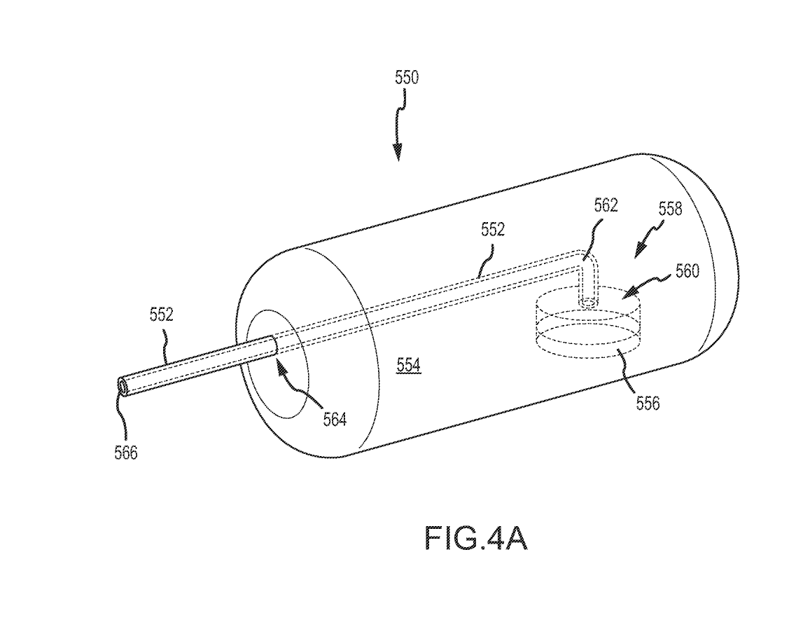

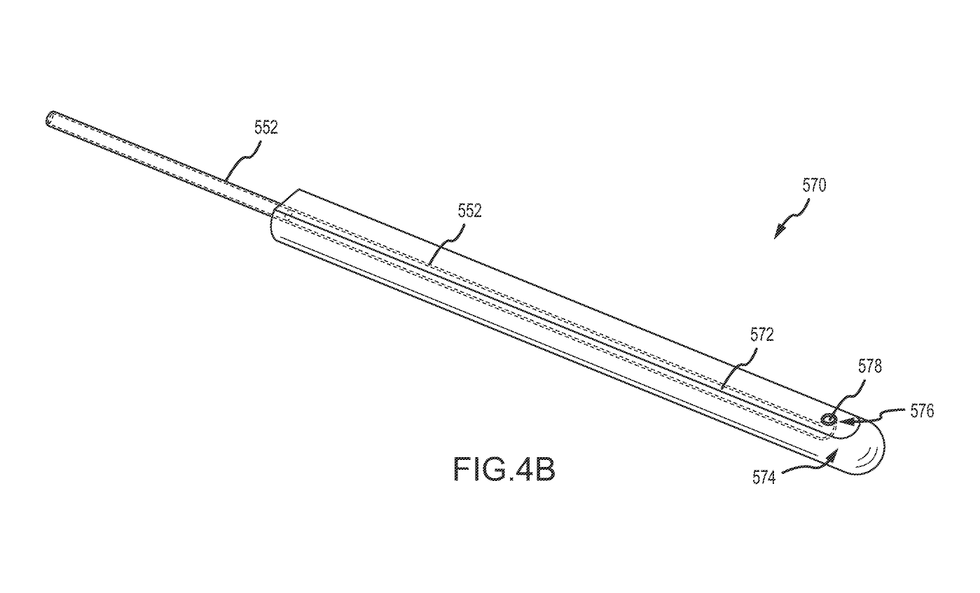

FIG. 4A depicts an embodiment of an adapter body 550 utilized in an internal component of an auditory prosthesis. The adapter 550 includes an outer surface 554 which can be any shape as required or desired for a particular application. Given the implantable nature of the adapter 550, however, low-profile shapes can be particularly desirable. Additionally, an elongate shape that provides a sufficient volume for the accommodation of a number of electrode contacts can also be desirable. The adapter 550 of FIG. 4A is simplified, and shows only a single electrode contact 556 substantially contained therein. A wire or other conductive element (not shown) connects the electrode contact 556 to the associated stimulator/receiver unit. The adapter 550 defines a void 558 that includes, in this embodiment, an electrode chamber 560 and a channel 562. In the depicted embodiment, the electrode chamber 560 is substantially cylindrical, but other geometries are contemplated. For example, the electrode chamber can be pyramidal, frustoconical, or conical in shape. In such embodiments, the electrode contact 556 is located proximate the wider base of the chamber 560. The electrode chamber 560 exposes a portion of the electrode contact 556 to the void 558. The channel 562 is connected to the electrode chamber 560 and terminates at an opening 564 defined by the outer surface 554 of the adapter 550. The depicted embodiment also includes an elongate structure 552 that extends from the adapter 550. The elongate structure 552 can have a circular, oval, square, or other cross-sectional shape. The elongate structure 552 is also disposed within the adapter 550 and terminates at the electrode chamber 560. Thus, an interior lumen of the elongate structure 552 is in fluidic communication with the electrode chamber 560. Alternatively, the elongate structure 552 can extend completely to the electrode 556. In other embodiments, the elongate structure 552 can penetrate the body 550 to a depth that enables the elongate structure 552 to be secured to the body 550. In the depicted embodiment, an end of the elongate structure 552 defines an electrical contact opening 566 when the void 558 and elongate structure 552 are filled with a conductive medium. This contact opening 566 acts as a stimulation site for a neural structure when the end of the elongate structure 552 is inserted directly into the cochlea. In other embodiments (described below), the elongate structure 552 terminates at an intracochlear body, which is inserted into the cochlea.

FIG. 4B depicts an embodiment of an intracochlear body 570 utilized in an internal component of an auditory prosthesis. Like the adapter 550 of FIG. 4A, the intracochlear body 570 is simplified, and shows only a single internal channel 572 formed therein. An outer surface 574 of the intracochlear body 570 defines an electrical contact opening 576 when the internal channel 572 is filled with a conductive medium. This contact opening 576 acts as a stimulation site for a neural structure when the intracochlear body 570 is inserted into the cochlea. In that regard, the intracochlear body 570 is typically elongate in shape. Existing commercially-available cochlear implants can have up to twenty-two electrode contacts to stimulate neural structures located within the cochlea. Accordingly, embodiments of the intracochlear bodies 570 described herein may include an equivalent number of contact openings 576. Of course, embodiments having any number of contact openings 576 are contemplated.

One or more elongate structures 552 can extend at least partially into the channels 572 of the intracochlear body 570. In certain embodiments, the elongate structures 552 terminate at the same or different distances into the intracochlear body 570. In other embodiments, the elongate structure 552 may extend completely to the contact opening 576. These elongate structures 552 can extend from the adapter 550 described above in FIG. 4A. Thus, signals output by stimulator are transmitted, via a conductive medium, to the intracochlear body 570. The conductive medium transmits the signals through the channel 572 of the intracochlear body 570 to a stimulation site (e.g., the contact opening 576).

In the depicted embodiment, the opening 576 is covered by a cover layer 578. The cover layer 578 can be used to retain a conductive medium within the channel 572, thus preventing leakage thereof. This can be useful in embodiments when the conductive medium is saline or other medical-grade fluid that is disposed within the adapter 550 and/or intracochlear body 570 prior to implantation. It should be noted that the adapter body 550 depicted in FIG. 4A can also utilize a cover layer at the contact opening 566 located at the end of the elongate structure 552. In order to ensure electrical signals sent from the electrode contact 556 (FIG. 4A) to the contact opening 576 are delivered to the targeted neural structure, in embodiments, it is desirable for the cover layer 578 to be made of a charge transfer material, such as TYVEK or TEFLON. Other suitable materials include polymeric materials, ionically conductive elastomers, or hydrogels such as polyacrylic acids, poly(meth)acrylic acids, polyalkylene oxides, polyvinyl alcohols, poly(N-vinyl lactams), polyacrylamides, poly(meth) acrylamides, or pressure sensitive adhesives such as a N-vinyl-pyrrolidone/acrylic acid copolymer. Additionally, the cover layer 578 need not be entirely solid, but can be of a mesh construction. Surface tension of the conductive medium contained within the channel 572 can be sufficient to prevent the conductive medium from leaking from the channel 572. In addition to preventing leakage of the conductive medium from the channel 572, utilization of a cover layer 578 can also prevent tissue ingrowth into the channel.

A cover layer is not required, however, to prevent certain embodiments of the adapter body 550 or intracochlear body 570 from retaining the conductive medium within the channel or elongate structure. In embodiments where the channel is of microtube or nanotube dimensions, surface tension of the conductive medium can prevent any fluid from leaking from the opening. For embodiments utilized in the above-described cochlear implants, channels and openings having cross-sectional areas of about 0.001 mm² to about 0.1 mm² are contemplated, as are cross-sectional areas of about 0.01 mm² to about 0.075 mm². In other embodiments, the cross-sectional area can be about 0.05 mm².

Additionally, there can be circumstances where it is desirable to encourage tissue growth into the contact opening, so as to ensure contact with the targeted neural structure. In such a case, the adapter body, the intracochlear body, and/or the elongate structure can include a cell growth factor or cytokine located proximate to the contact opening. Additionally, drugs such as dexamethasone or other classes of steroid drugs that have anti-inflammatory and/or immunosuppressant properties can be delivered via the devices described herein. In such an embodiment, an electrical charge can render a target cell wall porous, thus allowing the drug to enter. Gene therapies can be similarly delivered. Further, the bodies or elongate structures can be manufactured from materials that enable their use as reservoirs for active molecules such as medicaments, growth factors, or DNA. Additionally, the cover layer can serve to host and release, when appropriate, beneficial chemical and/or bioactive agents at the site of implantation of the flexible conductor. For example, anti-inflammatory, anti-bacterial, and/or anti-viral agents could be released from the cover layer. In another embodiment, cellular growth factors could be released from the cover layer.

Materials utilized in the flexible conductors described herein can be those that are biocompatible, flexible, robust, and that can be sterilized during or after manufacture. Flexible conductors include any of the adapters, elongate structures, microtubes, and intracochlear bodies that include a hollow structure adapted to receive a conductive medium. In embodiments, materials that stretch without deformation can be used. Examples of materials that can be utilized for the adapter body include silicone elastomeric material such as Silastic material, polyamide, PVC, polyurethane blends, or other types of polymers or elastomers that are typically used for implantable insulators. The elongate structure and intracochlear body can be manufactured from similar materials. Additionally, electrically conductive materials such as polymeric materials, ionically conductive elastomers, or hydrogels such as polyacrylic acids, poly(meth)acrylic acids, polyalkylene oxides, polyvinyl alcohols, poly(N-vinyl lactams), polyacrylamides, poly(meth) acrylamides, or pressure sensitive adhesives such as a N-vinyl-pyrrolidone/acrylic acid copolymer can also be utilized. Suitable materials for electrode contacts include platinum, stable platinum iridium, or other highly conductive metals or conductive plastics. Of course, flexible conductors that are not utilized within a human or mammalian body can utilize different types of materials.

Existing implantable conductors incorporate a metallic structure such as a wire to act as an electrical conductor and metallic surfaces to deliver charge to a neuron. Typically, these metallic elements are embedded in the softer flexible elastomer. Thus, the finished component may not be as flexible as desired. To address these and other issues, the flexible conductors described herein utilize highly deformable conductive media to transfer electrical signals from the electrode contact to a target neuron in a cochlea. In certain embodiments, the conductive media is characterized by a viscosity. Examples of such media include liquids, fluids, colloids, suspensions, or solutions. Mobile fluids and viscous fluids can be utilized. The conductive media can include discrete metallic structures, such as carbon nanotubes, to increase electrical conductivity. The conductive media can also be metallic or ionic. In certain embodiments, saline is used. Additionally, the flexible conductors described herein can be further configured such that body fluids located in the area in which the flexible conductor is implanted can be drawn into the void and/or the channel to serve as the conductive medium. For example, the void can contain, or have disposed thereon, a hydrophilic material that facilitates the drawing of the desired fluid into the body. In embodiments where the interior void is defined by a channel and an electrode chamber, the hydrophilic material can be disposed in one or both of those structures. Certain embodiments include hydrophilic material within the entire void and/or channel, so as to facilitate the drawing of the desired fluid into contact with an electrode. In certain embodiments, the elongate structure itself can be formed of a hydrophilic material so as absorb the desired fluid. In addition to holding the fluid in a hydrophilic material of the elongate structure, the elongate structure can be constructed of material that otherwise promotes transfer of electrical charge along the walls thereof. Bodily fluids that display sufficient conductivity for particular applications include cerebral spinal fluid, perilymph, blood, and others.

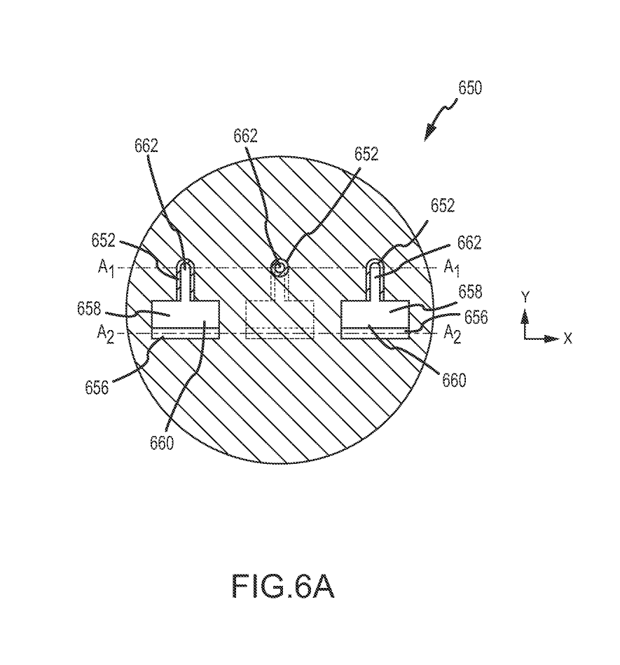

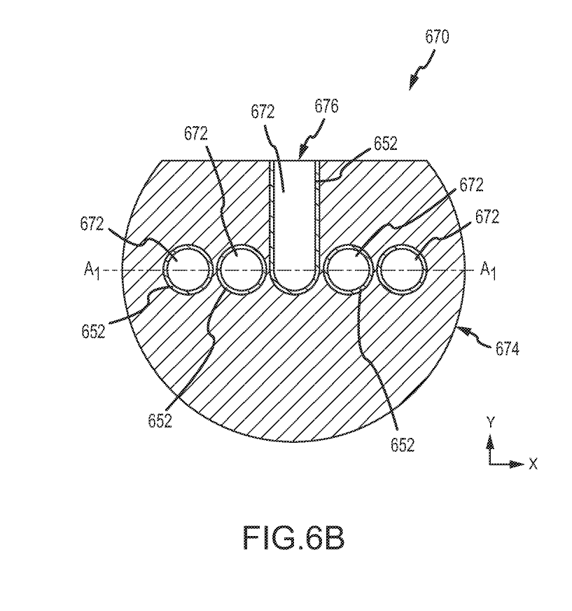

FIG. 5 depicts a partial top view of another embodiment of an internal component 644 of an auditory prosthesis. Additionally, FIGS. 6A and 6B depict cross-sectional views of an adapter body 650 and an intracochlear body 670, respectively. Accordingly, FIGS. 5-6B are described simultaneously. Here, multiple electrode contacts 656 are embedded within the adapter 650. Each electrode contact 656 is exposed to a void 658. In the depicted embodiment, the portion of the void 658 proximate the electrode contact 656 is an electrode chamber 660 sized to expose nearly the entire surface area of the electrode contact 656, though exposure of smaller areas of the electrode contact 656 is also contemplated. The electrode chamber 660 is in fluidic communication with a channel 662 defined by the body 650. In this embodiment, an elongate structure 652 is disposed within the channel 662 and terminates, at one end, at the electrode chamber 660. The elongate structure 652 extends through the channel 662 and out of an opening 664 defined by an outer surface 654 of the adapter 650. Each elongate structure 652 connects to the intracochlear body 670. In the depicted embodiment, the elongate structures 652 are bundled into a shape and size consistent with that of the intracochlear body 670.

The intracochlear body 670 also includes a plurality of internal channels 672 formed therein. The elongate structures 652 can extend through the channels 672 and terminate at an outer surface 674 of the intracochlear body 670. Each channel 672 in the intracochlear body 670 terminates at the outer surface 674 thereof, at a contact opening 676. Each contact opening 676 acts as a stimulation site that is used to stimulate a neural structure within the cochlea, once the intracochlear body 670 is implanted therein. Signals output by the electrode 656 propogate through the conductive medium as described above with regard to FIGS. 4A and 4B. Although the depicted embodiment depicts an elongate structure 652 extending through both the channel 662 and the channel 672, other embodiments need not utilize such elongate structures.

FIGS. 5-6B depict the elongate structures 652 and channels 662, 672 generally disposed along a single linear axis Al. This depiction is for clarity only. Orientation and spacing of the elongate structures 652 and the channels 662, 672 can be as desired along either of the x-axis or y-axis so as to conserve space within the adapter or intracochlear body 670. Additionally, the electrode contacts 656 need not be disposed along an axis A2, as depicted in FIG. 6A. Instead, the electrode contacts 656 can be disposed and oriented within the adapter 650 based on space considerations, contact area optimization, or other factors. Similarly, contact openings 676 can be arranged in any orientation to form a contact array. In certain embodiments, a single channel 662, 672 can be in fluidic communication with a plurality of electrodes, and thus be able to transmit electrical signals from more than a single electrode contact.

This disclosure described some embodiments of the present technology with reference to the accompanying drawings, in which only some of the possible embodiments were shown. Other aspects can, however, be embodied in many different forms and should not be construed as limited to the embodiments set forth herein. Rather, these embodiments were provided so that this disclosure was thorough and complete and fully conveyed the scope of the possible embodiments to those skilled in the art.

Although specific embodiments were described herein, the scope of the technology is not limited to those specific embodiments. One skilled in the art will recognize other embodiments or improvements that are within the scope of the present technology. Therefore, the specific structure, acts, or media are disclosed only as illustrative embodiments. The scope of the technology is defined by the following claims and any equivalents therein.

What is claimed is:

1. An apparatus comprising a body comprising an exterior surface, wherein the exterior surface at least partially defines an opening, and wherein the body at least partially defines an interior channel extending into the body from the opening, and wherein the interior channel is adapted to receive a conductive medium.

2. The apparatus of claim 1, further comprising the conductive medium, wherein the conductive medium located proximate the opening comprises a stimulation site.

3. The apparatus of claim 2, wherein the conductive medium comprises at least one of a liquid, a fluid, a colloid, a suspension, and a solution.

4. The apparatus of claim 2, wherein the conductive medium further comprises carbon nanotube particles.

5. The apparatus of claim 2, further comprising a cover layer covering the opening so as to retain the conductive medium within the interior channel, wherein the cover layer comprises at least one of a charge transfer material and an ionically conductive material.

6. The apparatus of claim 1, wherein at least a portion of a surface of the interior channel is substantially hydrophilic.

7. The apparatus of claim 1, wherein the body comprises a substantially elongate structure.

8. The apparatus of claim 2, wherein at least one of the conductive medium and the interior channel comprise a microtube.

9. The apparatus of claim 1, further comprising an electrode contact disposed within the body, wherein at least a portion of the electrode contact is exposed to conductive medium.

10. An apparatus comprising an elongate body defining an interior channel, wherein the interior void is adapted to receive an electrically conductive medium displaying a viscosity.

11. The apparatus of claim 10, further comprising a microtube disposed within the interior channel and adapted to receive the conductive medium.

12. The apparatus of claim 11, wherein the microtube extends from the elongate body.

13. The apparatus of claim 10, wherein the interior void terminates at a stimulation site defined by an exterior surface of the elongate body, and wherein the apparatus further comprises a cover disposed so as to cover the opening.

14. The apparatus of claim 10, further comprising an electrode contact disposed within the body, wherein at least a portion of the electrode contact is exposed to the conductive medium.

15. An apparatus comprising:
an implantable stimulation unit;
a body connected to the implantable stimulation unit, the body defining at least one interior channel adapted to receive a conductive medium displaying a viscosity; and
contact operatively connected to the stimulation unit.

16. The apparatus of claim 15, wherein the body is fixed to the implantable stimulation unit.

17. The apparatus of claim 15, wherein the body is located discrete from the implantable stimulation unit and wherein the body is connected to the implantable stimulation unit with a lead.

18. The apparatus of claim 15, further comprising an elongate structure adapted to receive the conductive medium.

19. The apparatus of claim 18, wherein the elongate structure is at least partially disposed within the body.

20. The apparatus of claim 15, wherein at least a portion of a surface of the interior channel is substantially hydrophilic.
